(19) United States
(12) Patent Application Publication
(10) Pub. No.: US 2011/0270168 A1
(43) Pub. Date: Nov. 3, 2011

(54) RELEASING DEVICE FOR ADMINISTERING A BIO-ACTIVE AGENT

(76) Inventors: Izhar Halahmi, Hod Hasharon (IL); Guy Ben Simon, Tel Aviv (IL); Offer Fabian, Tzur Moshe (IL)

(21) Appl. No.: 13/099,583

(22) Filed: May 3, 2011

Related U.S. Application Data
(60) Provisional application No. 61/330,560, filed on May 3, 2010.

(51) Int. Cl. A61M 5/00; B05D 3/12; B05D 3/10; C25D 5/54; B05D 5/00

(52) U.S. Cl. 604/93.01; 205/159; 427/2.21; 216/37

(57) ABSTRACT

A releasing device for administering a bio-active agent to a human, animal, or plant is described herein. The device comprises a core of a solid organic matrix, in which a bio-active agent is contained. A metallic layer surrounds the core to form an outer shell, and at least one aperture is present in the metallic layer for controlled release of the bio-active agent to the patient. Optionally, a third, polymeric, ceramic or organo-ceramic layer is provided, and may be loaded by bioactive agent as well.

### CROSS-REFERENCE TO RELATED APPLICATIONS

This application claims priority to U.S. Provisional Patent Application Ser. No. 61/330,560, filed May 3, 2010. That application is hereby fully incorporated by reference herein.

### BACKGROUND

Miniature devices that can be implanted, placed, or attached to the organ of a human, animal, or plant, then slowly release a bio-active agent (referred to herein as BAA), have been used for many years and are under continuous development around the world.

Such devices that are constructed using polymers are low-cost, bio-stable or biodegradable, lightweight and compatible with many bio-active agents. However, use of polymers has a major drawback—especially when long term release is required: their relatively high permeability. Some polymers, such as polytetrafluoroethylene (PTFE), are relatively impermeable, but are also difficult to process, have weak structural integrity, and are soft. Practically, a device constructed from, or encapsulated by, such polymers may not be useful.

Metals are generally hard, have a higher degree of structural integrity and are relatively impermeable, but their processing methods limit their use to relatively large and robust devices.

There is thus a need to enable manufacturing of small, lightweight devices, that are resistant to puncture, impregnation, etc.; can be loaded with a bio-active agent; and are characterized by a slow release rate of the BAA to the surrounding biological medium abutting the device. Such biological medium includes, but is not limited to, a body organ such as blood, tissue, fat, liver, muscle, eye fluid, and the like, as well as animal and plant tissues. Preferably, the BAA can be released for a period of three months or more without the need to reload the device with additional BAA.

### BRIEF DESCRIPTION

The present disclosure relates to medical devices for administering a bio-active agent which can be implanted or placed into the body of a human or an animal patient or in any kind of living tissue, including tissue cultures and plants. The device comprises a core and a shell. The core contains a bio-active agent which is incorporated within a liquid, gel, powder, solid, or heterogeneous matrix. The matrix is a solid organic compound. Exemplary matrix materials include fibrous matter, nanoparticles, nanofibers, nanotubes, and crosslinked or non-crosslinked polymers. The matrix may also be mixed with particles, fibers, liquids, or gas voids. In a preferred embodiment, the core comprises a polymeric binder. The shell is metallic, and can be shaped as desired, or its hermeticity (i.e. permeability) can be tailored as desired, to achieve a pre-determined release rate of the bio-active agent from the device to the patient. The shell comprises one or more metallic layers, made of the same or different materials.

Disclosed in certain embodiments is a releasing device for administering a bio-active agent to a patient or organ or tissue, the device comprising: a core including a solid organic matrix and a bio-active agent contained within the solid organic matrix; and a metallic layer surrounding the core; wherein the core is exposed to its surrounding environment of the patient or organ or tissue through at least one aperture in the metallic layer. The term “solid organic” matrix refers to a gel, wax, oligomer, polymer, cross-linked polymer, and polymeric materials comprising carbon and hydrogen atoms as well as possibly other atoms such as silicon, zirconium, titanium, or aluminum. The bio-active agent may be dissolved in the organic matrix, adsorbed to a second organic or inorganic phase in the matrix, dispersed in a second organic phase in the matrix, and combinations thereof.

In some embodiments, the bio-active agent is first adsorbed to particles made from materials such as metal oxide, metal carbonate, metal phosphate, metal sulfate, glass, or ceramic. After adsorption, the particles are dispersed in the organic matrix. The bio-active agent may dissolve in the organic matrix or remain on the dispersed particles.

Also disclosed is a process for making a releasing device for a bio-active agent, comprising: forming a core having a solid organic matrix containing the bio-active agent; applying a metal or metallic layer to the core to form an outer shell; and forming at least one aperture in the metallic layer.

The core can be formed by: mixing the bio-active agent with a polymer, a monomer or oligomer solution, emulsion, melt, or dispersion that undergoes crosslinking or polymerization, a molten wax or polymer that undergoes a solidification, or a polymer or oligomer or wax powder that is later sintered under pressure and/or heat to form a mixture; and shaping the mixture into a desired shape for the core.

Alternatively, the core can be formed by adsorbing the bio-active agent onto particles made from materials such as metal oxide, metal carbonate, metal phosphate, metal sulfate, glass, or ceramic. After adsorption, the particles are dispersed in the organic matrix. In yet another method, the bio-active agent is mixed with a polymer, a monomer or oligomer solution, emulsion, melt or dispersion that undergoes crosslinking or polymerization, a molten wax or polymer that undergoes a solidification, or a polymer or oligomer or wax powder. This combination is then sintered under pressure and/or heat to form a mixture. The mixture can then be shaped into a desired shape for the core.

The metallic layer can be applied by a vapor phase process (such as evaporation or sputtering), chemical vapor deposition, an electroless process, or an electroplating process, as well as combinations thereof to form an exterior shell or encasement. The device may in some embodiments comprise two or more metallic layers, with each layer being made of the same or different metals.

The at least one aperture can be formed by milling, abrading, abrading, etching, or drilling of the metallic layer. Alternatively, the at least one aperture can be formed by applying an etch resist to the metallic layer, etching at least one aperture into the metallic layer, and removing the etch resist. Also, the at least one aperture can be formed by applying at least one plating resist to the core, applying a metallic layer to the core, and removing the at least one plating resist to form the at least one aperture in the metallic layer.

These and other non-limiting characteristics of the disclosure are more particularly disclosed below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 provides a schematic drawing of the core of he device, with dissolved or dispersed bio-active agent in a solid organic matrix.

FIG. 2 provides a schematic drawing of a core surrounded by a metal shell or outer layer.

FIGS. 3A-3C show schematic drawings of a core surrounded by a metallic layer, wherein at least one edge and/or at least one face is milled or abraded, so that a portion of the metallic layer is removed. By controlling the degree of metal removal, the release rate of the bio-active agent can be well-controlled.

FIG. 4 provides a schematic drawing of a core surrounded by a metallic layer, wherein at least one edge and/or at least one face is selectively etched by wet or dry etching, so that a portion of the metallic layer is removed. By controlling the degree of metal removal, the release rate of the bio-active agent can be well-controlled.

FIG. 5 provides a schematic drawing of a core surrounded by a metallic layer, wherein at least one edge and/or at least one face is selectively drilled or punched, so that a portion of the metallic layer is removed. By controlling the degree of metal removal, the release rate of the bio-active agent can be well-controlled.

FIG. 6 provides a schematic drawing of a core surrounded by a metallic layer, wherein at least one edge and/or at least one face is selectively plated, so that a portion of the metallic layer is removed. By controlling the degree of metal removal, the release rate of the bio-active agent can be well-controlled.

### DETAILED DESCRIPTION

A more complete understanding of the components, processes, and apparatuses disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range of “from about 2 to about 10” also discloses the range “from 2 to 10.”

The term “comprising” is used herein in an open-ended sense that requires at least the listed components and does not exclude the presence of other unspecified components. For example, a device comprising components A, B, and C can also have component D. The term “comprising” should also be construed as disclosing devices that “consist of” the listed components, i.e. only the listed components are allowed, and the presence of other unspecified components is excluded.

The present disclosure provides a releasing device which is suitable for the delivery of bio-active agents, such as drugs, hormones, minerals, radioactive materials, DNA, RNA, bacteria, viruses, fungi, toxins, neurotransmitters, stimulators, inhibitors, neurotransmitters, precursors of functional molecules, or other bio-active compounds or molecules for any known living tissue. The term “living tissue” refers to any biological cell plurality that has an active metabolism. Exemplary living tissues include mammalian tissues, other eukaryotes, prokaryotes, plants, fungi and bacteria. Non limiting examples of useful applications are sub-conjunctival, intra-ocular, intra-cranial, intra-thecal, epidural, sub-dural, or within a living organ or tissue. Such organs or tissues may include any within the central nervous system, reproductive organs, kidneys, the liver, bones, the lymph system, blood, intestine, pancreas, or within plant tissues. The devices comprise a core and at least one metallic layer. The metallic layer can also be considered a shell or an outer layer of the overall device, relative to the core.

In preferred embodiments, the metallic layer has a thickness of from about 0.01 micrometers to about 100 micrometers. In more specific embodiments, the metallic layer has a thickness of from about 0.5 micrometers to about 50 micrometers, or a thickness of from about 1 micrometers to about 25 micrometers.

The metallic layer provides the device with some novel and unique properties.

First, the metallic layer forms an impermeable barrier—unlike organic matrices such as polymers that are relatively permeable, metals are absolutely impermeable. For example, a nickel layer of 0.5 micrometers thickness is more impermeable (i.e. less permeable) than a 1 millimeter thick layer of PTFE or a 10 millimeter thick layer of polyethylene to relatively small molecules and compounds as will be further discussed herein.

Second, the metallic layer is scratch resistant, corrosion resistant, bio-inert, resistant to hydrolysis and swelling, strong, and ductile.

Third, the metallic layer can be biocompatible when certain metals are used, such as gold, silver, platinum, stainless steel, or titanium.

Fourth, the metallic layer can be easily applied to the core to form a thin layer that is free of pinholes. In contrast, polymeric layers are subject to defects and pinholes when they are thin layers (i.e. thinner than 10 micrometers).

In preferred embodiments, the metallic layer itself comprises an inner layer and an outer layer. The inner layer is closer to the core than the outer layer of the metallic layer. In specific embodiments, the outer layer is a noble metal, such as gold or platinum, that provides the device with the required biostability and inertness towards body tissues or living tissues.

In some embodiment, an outer surface of the metallic layer is surrounded by a passivating layer to passivate the metallic layer. This passivating layer may be made from glass, a ceramic, or an organo-ceramic. The passivating layer is typically deposited by a sol-gel or chemical vapor deposition process.

The metallic layer surrounds the core. The core, or core layer, comprises an organic solid matrix. The matrix can be thermoplastic or thermosetting (cross-linked). A bio-active agent is contained within the solid organic matrix. The bio-active agent can be dissolved, dispersed, emulsified, bound, adsorbed, impregnated, mixed, or otherwise placed into the solid organic matrix. The bio-active agent may be directly mixed in with the organic matrix. Alternatively, the bio-active agent may be adsorbed to another material, usually a particulate or fibrous matter, which is then mixed in with the organic matrix.

In preferred embodiments, the bio-active agent is first dissolved, dispersed or emulsified into a organic compound (or its precursors) melt, solution, emulsion or dispersion. Typical compounds useful for the solid organic matrix are polymers, oligomers, monomers, wax, oils, plasticizers, and combinations thereof. The resulting mixture is then solidified into the final shape for the core, or solidified into an intermediate shape. The intermediate shape can then be processed to form the final shape for the core.

In other preferred embodiments, the bio-active agent is first dissolved, emulsified, or dispersed in a volatile medium. The bio-active agent is then mixed with particles of a material capable of adsorbing the bio-active agent to form a mixture. Such adsorbent materials may include, for example, metal oxide, metal carbonate, metal phosphate, metal sulfate, glass, ceramic, cellulose derivatives and polysaccharides such as chitosan. The mixture is dried, so the particles are coated, impregnated, and/or adsorbed with the bio-active agent. The bio-active agent loaded particles are then dispersed in the organic matrix solution. The mixture is then solidified through drying, curing, crystallization, gelation, vitrifying, cross-linking, and/or polymerization. The solid mass is shaped to the desired shape in a mold or post-molding.

The solid organic matrix of the core is formed from one or more compounds or precursors that are suitable for long-term contact with living tissues. In one embodiment, the device is suitable for implanting or placement or swallowing or abutting an biological medium or a body organ, such as in the human body, as well as animal or plant tissues. By “long-term” is meant a continuous period of at least 3 months and more preferably at least one year. In a preferred embodiment, said device is designed for release for periods greater than 2 years, and may be very useful for treating chronic diseases such as diabetes, glaucoma, autoimmune diseases, cancer, AIDS, etc. Exemplary polymers include, but are not limited to, poly(dimethylsiloxane), polyurethanes, epoxies, methyl methacrylate polymers, acrylic copolymers, polyesters, polyamides, polyethylene, polypropylene, ethylene copolymers and terpolymers, propylene copolymers and terpolymers, fluoropolymers, vinyls, styrenics, polycarbonates, amino resins, and phenolic resins. Other exemplary polymers include crosslinked acrylic or methacrylic networks, including networks formed by ultraviolet (UV) curing. In selected embodiments, the core comprises a thermosetting polymer. Exemplary waxes include, but are not limited to, paraffins, amides, esters, fatty acid derivatives, fatty alcohol derivatives, silicones, and phospholipids.

Generally, the releasing device and the core are considered to have a cylindrical shape, a cubical shape, a box shape, or a “coin” shape. The length of the core may vary from about 0.1 mm to about 50 mm. The width, thickness, or diameter of the core in a cylindrical shape may vary from about 10 micrometers to about 10 millimeters. The diameter of the core having a coin-like shape may vary from about 0.1 mm to about 50 mm.

In preferred embodiments, the core is designed for ocular inclusion, i.e. the device is placed at or near the ocular, ocular adnexa and orbital tissue. The length of the device is in the range of from about 1 mm to about 10 mm. The diameter of the device is from about 0.025 mm to about 5 mm.

A bio-active agent is contained within the polymeric matrix. The term “bio-active agent” refers to a molecule or compound that causes a direct biological reaction to the molecule or compound or products thereof (i.e. the bio-active agent is a precursor of a molecule or compound that causes a biological reaction). The biological reaction can be from the human/animal/bacteria/virus/fungus/plant in which or near which the device has been implanted or placed, or from some other organism living inside the human/animal. Exemplary bio-active agents include, but are not limited to: anti-glaucoma medications or any combination of anti-glaucoma medications, including prostanglandin analogues, beta blockers, alpha agonists, carbonic anhydrase inhibitors, and miotics; anti-inflammatory medications such as steroids, non-steroidal anti-inflammatory drugs (NSAIDs), and Cox-2 inhibitors; pro-inflammatory medications; immune modulating drugs; chemotherapeutic drugs like antineoplastics; antibiotic agents; anti-viral drugs; anti-fungal drugs; anti-VEGF (vascular endothelial growth factors) drugs; mydriatics; hormones; vitamins; minerals; radioactive agents; and toxins.

The bio-active agent is generally distributed within the solid organic matrix to provide a relatively constant release rate of the bio-active agent through the metallic layer. This distribution also affects the mechanical integrity, ease of handling, and manufacturing ease of the core.

The metallic layer(s) can be deposited onto the core using known processes, such as: (a) vapor phase processes, such as sputtering, evaporation, chemical vapor deposition, physical vapor deposition, and plasma assisted deposition; (b) electroless plating processes, for metals such as palladium, tin, silver, copper, and nickel; and (c) electroplating processes. The adhesion of the metallic layer(s) to the core may be enhanced by various etching processes. Those etching processes may be physical etching processes, such as plasma, corona, flame, sand blasting, or chemical-mechanical polishing; or wet chemical etching processes using oxidizing agents such as permanganate or hypochlorite.

The release rate of the bio-active agent from the device can be controlled by: (a) control of the metallic layer thickness; and (b) perforation or selective removal of portions of the metallic layer by (1) selective plating, (2) mechanical, chemical, laser or plasma drilling, (3) selective chemical etching, (4) milling or abrading on an edge, corner, or face of the device, and/or (5) mechanical punching. This results in the formation of holes or apertures in the metallic layer, which expose portions of the core and allow the bio-active agent to be released from the core into the ambient environment. Another method of forming pre-defined apertures in the metallic layer is by selective plating or deposition of said metal through a mask or resist onto said core.

In preferred embodiments, the metal outer layer is milled or eroded, so that a portion of the core is exposed. The ratio of the milled area (i.e. the removed area) to the total surface area of the device can vary from 0.01% to 90%, more specifically from 0.25% to 25%, and even 1% to 15%.

In other preferred embodiments, the metal outer layer is perforated, drilled, or punched, so that a portion of the core is exposed. The ratio of the perforated or punched areas to the total surface area of the device can vary from 0.01% to 90%, more specifically from 0.25% to 25%, and even 1% to 15%.

Also provided are processes for making a device for the slow release of bio-active agent. Those processes generally comprise the steps of forming a core, applying a metallic layer to the core, and optionally encapsulating the resulting device with an external layer. Said external layer can be polymeric, ceramic, glass, or organo-ceramic (ceramer). In preferred embodiments, said external layer is loaded with at least one bio-active molecule. In one selected embodiment, said device is implanted or placed in a human or animal body, and said bio-active agent in said external layer is an anti-inflammatory or an immune system inhibitor.

The metal outer layer may be a relatively low cost metal such as copper, chrome, or nickel for living tissues that are not subjected to strict regulations (plants, kinds of animals, tissue cultures), or from more expensive noble metals which are inert and bio-compatible with animal/human tissue. In order to provide an economical device, an outer layer of a noble metal, deposited on at least one inner layer of a lower cost metal, is disclosed.

As discussed above, the bio-active agent can be dissolved, dispersed, or emulsified with the solid organic matrix. The matrix itself can be processed in the form of a melt, emulsion, dispersion or solution, i.e. already liquefied, when mixed with the bio-active agent. Alternatively, depending on the stability of the bio-active agent, the polymer can be in a solid form such as powder, flakes, or fibers, when mixed with the bio-active agent. The solid mixture can then be densified, for example by the application of heat, pressure, sound waves, infrared irradiation, UV light, and combinations thereof.

The mixture of polymer and the at least one bio-active agent is then solidified into a solid, self-supporting shape. This shape can be the desired shape of the overall device, or the solid core can be processed, such as by trimming or cutting, into the desired shape. Typical useful shapes are cylinder, coin, disk, plate, cube, sphere, fiber, box, diamond, ring, “S”, “L”, “T”, web, net, mesh, “U”, or “V”.

The outer layer of the core can optionally be activated for metallization prior to the metallization. Activation refers to etching and/or roughening the surface to provide better adhesion between the organic core and the metallic shell. Exemplary processes useful for activation are plasma and corona etching; flame treatment; immersion in oxidizers such as permanganate, hypochlorite, or hydrogen peroxide; and blasting with abrasive particles.

Next, the core is metallized, or in other words the metallic layer is applied to the core to form a shell or metal skin. If desired, more than one metallic layer can be applied to the core. The metallization process can be performed in a gaseous state, for example evaporation and sputtering, or a liquid state such as electroless metallization. Metal thickness can be increased by electroplating. In a preferred embodiment, the metal shell comprises one layer of a noble metal, with a total of two or more layers, wherein the outer metallic layer is the noble metal.

Then, the metallic layer can be processed to provide a device having a controlled and pre-determined release rate for the bio-active agent. The metallic layer can be processed by, for example, milling, perforating, punching, or etching the metallic layer so that some portion of the core is exposed through holes or apertures. Put another way, the bio-active drug can travel from the core through the metallic layer into the ambient environment.

In some embodiments, the device is useful as a medical device, comprising the core and the metallic shell, and can be optionally encapsulated with one or more polymeric, ceramic or organo-ceramic layers, also known as encapsulating layers. The role of the encapsulating layers is to provide mechanical protection—especially when the metal shell is thin, to increase biocompatibility, to provide lubricity, to delay inflammatory reactions toward said device, and to enable color coding and labeling.

In preferred embodiments, at least one encapsulating layer comprises at least one bio-active agent, such as drug. The term “drug” refers to any compound that affects living tissue by causing a biological reaction. For example, the drug may assist in healing, lowering pain, deactivating pathogens, inhibiting scar tissue growth, inhibiting inflammatory processes, reducing intra-ocular pressure (IOP), inhibiting the formation of new blood vessels, decreasing vascular permeability, increasing tears formation, increasing ocular moisture, modulating immune response, and contracting or dilating the pupil of the eye, if the device is used for ocular therapy.

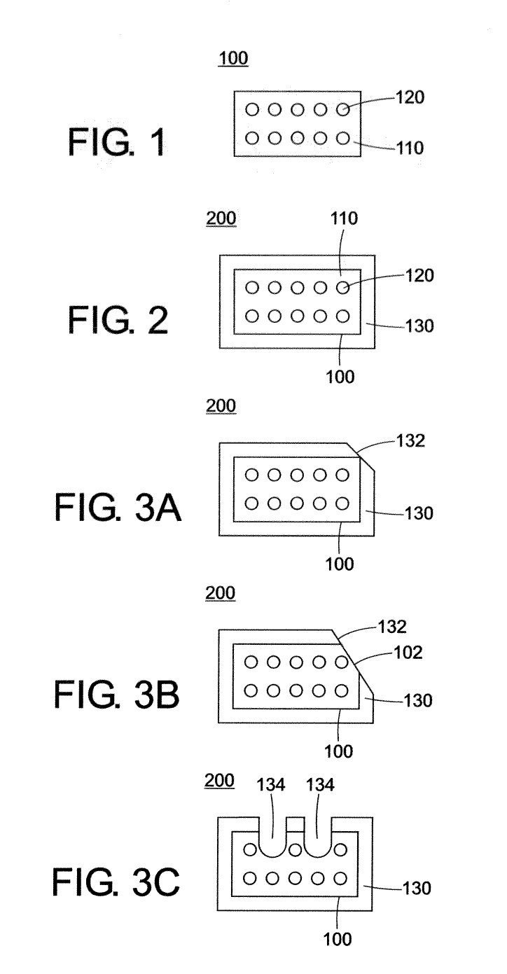

FIG. 1 is a cross-sectional view of the core 100. As noted above, the core has a generally cylindrical or box shape when viewed in three dimensions, and this view is the longitudinal cross-section. The core is formed from an organic matrix core 110 that contains a bio-active agent 120 (BAA).

FIG. 2 is a cross-sectional view of the completed device 200. The core 100 is surrounded by a metallic layer 130.

FIGS. 3A-3C illustrate different locations and shapes of apertures in the metallic layer. In FIG. 3A, a corner of the metallic layer has been shaved off to form an aperture 132. In FIG. 3B, a larger portion of the corner of the metallic layer is shaved off, as well as a small portion of the core itself (reference numeral 102). This changes the surface area of the core that is exposed through the aperture 132. Finally, in FIG. 3C, two separate apertures 134 are shown in the metallic layer 130. In addition, the core 100 has a semicircular shape beneath each aperture 134. This shaping of the core can be performed prior to application of the metallic layer, or afterwards in conjunction with the formation of the apertures in the metallic layer.

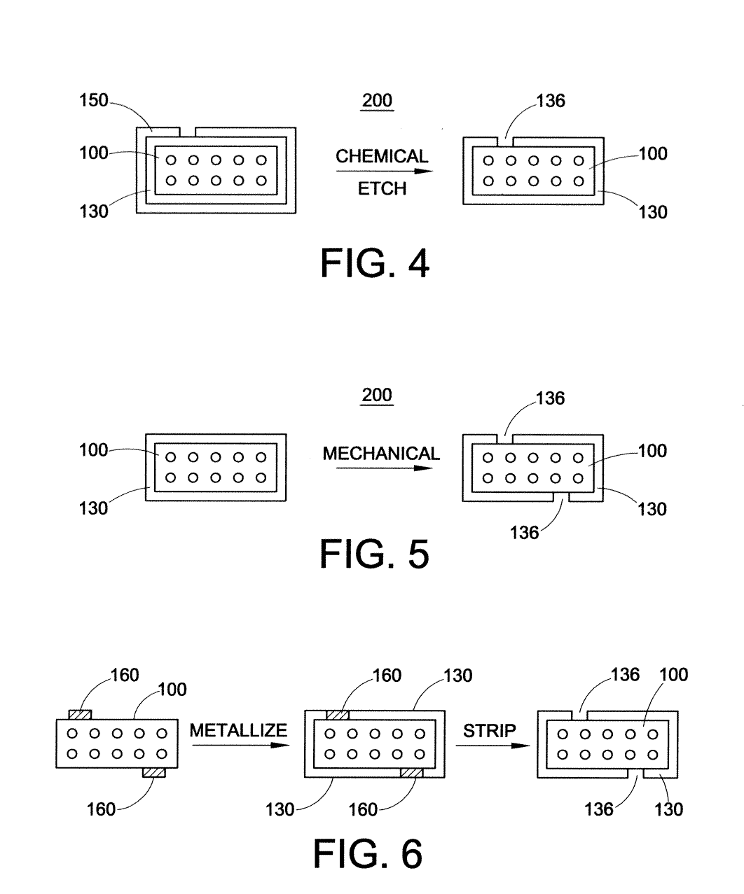

FIG. 4 illustrates one method of forming apertures in the metallic layer. As depicted here, an etch resist 150 is placed on the metallic layer 130. The etch resist resists etching from the particular material used to remove the metal, allowing for selective etching of the exposed portions that are not covered by the etch resist. The etch resist is then removed to obtain the device 200 with aperture 136.

FIG. 5 illustrates another method of forming apertures in the metallic layer 130. Here, apertures are formed by using a laser, mechanical drill, or punch to form the apertures 136.

FIG. 6 illustrates yet another method of forming apertures in the metallic layer. Here, a plating resist 160 is located on the core 100 prior to applying the metallic layer 130. Metal is then applied to the uncovered areas, which forms the metallic layer 130 on the core 100 except where the plating resist 160 is located. The plating resist 160 is then stripped to form the apertures 136.

The bio-active agent in the core, particularly when the bio-active agent is a pharmaceutical compound, can be in the form of pharmaceutically acceptable salts derived from inorganic or organic acids, in combination with one or more pharmaceutically acceptable excipients. The phrase “pharmaceutically acceptable salt” means those salts which are within the scope of sound medical judgment, suitable for use in contact with the tissues without undue toxicity, irritation, allergic response, and the like, and are commensurate with a reasonable benefit/risk ratio. Pharmaceutically acceptable salts are well known in the art. The salts can be prepared either in situ during the final isolation and purification of the bio-active agent, or separately by reacting the acidic or basic drug substance with a suitable base or acid respectively. Typical salts derived from organic or inorganic acids salts include, but are not limited to hydrochloride, hydrobromide, hydroiodide, acetate, adipate, alginate, citrate, aspartate, benzoate, bisulfate, gluconate, fumarate, hydroiodide, lactate, maleate, oxalate, palmitoate, pectinate, succinate, tartrate, phosphate, glutamate, and bicarbonate. Typical salts derived from organic or inorganic bases include, but are not limited to lithium, sodium, potassium, calcium, magnesium, ammonium, monoalkylammonium such as meglumine, dialkylammonium, trialkylammonium, and tetralkylammonium.

It is contemplated that the medical device can be used to administer the bio-active agent in many different ways depending on how and where it is implanted or placed. For example, the mode of administration of the bio-active agent can be intravenous, intramuscular, intracisternal, intraperitoneal, subcutaneous, or intrasternal.

Actual dosage levels of the active ingredients of the bio-active agent can be varied in order to achieve the effective therapeutic response for a particular patient. The phrase “therapeutically effective amount” means a sufficient amount of the compound to treat disorders, at a reasonable benefit/risk ratio applicable to any medical treatment. The specific therapeutically effective dose level for any particular patient will depend upon a variety of factors including the disorder being treated and the severity of the disorder; medical history of the patient, activity of the specific bio-agent employed; the specific composition employed, age, body weight, general health, sex and diet of the patient, the duration of the treatment, rate of excretion of the bio-active agent, drugs used in combination or coincidental with the bio-active agent; and the like. It is contemplated, however, that the medical device maintains a constant release rate over a long-term period, i.e. at least three months.

The bio-active agent can be formulated together with one or more non-toxic pharmaceutically acceptable diluents, carriers, adjuvants, and antibacterial and antifungal agents such as parabens, chlorobutanol, phenol, sorbic acid, and the like. The bio-active agent itself can be modified to change its release rate, for example by suspending the bio-active agent in a vehicle having poor water solubility such as oils.

Other materials may be present in the core as well, such as physiologically acceptable, isotonic sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. The core can also contain adjuvants such as preserving, wetting, emulsifying, and dispensing agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, or mixtures of these substances.

The bio-active agent can be considered to be microencapsuled in the matrix of a biodegradable polymer, such as polylactide-polyglycolide. Depending upon the ratio of bio-active to polymer and the nature of the particular polymer employed, the rate of release can also be controlled. Examples of other biodegradable polymers include poly(orthoesters) and poly(anhydrides). The bio-active agent could also be made in the form of liposomes or microemulsions that are compatible with body tissues.

The bio-active agent may be mixed with at least one inert, pharmaceutically acceptable excipient or carrier, such as sodium citrate or dicalcium phosphate and/or (a) fillers or extenders such as starches, lactose, sucrose, glucose, mannitol and silicic acid; (b) binders such as carboxymethylcellulose, alginates, gelatin, polyvinylpyrrolidone, sucrose and acacia; (c) humectants such as glycerol; (d) disintegrating agents such as agar-agar, calcium carbonate, potato or tapioca starch, alginic acid, certain silicates and sodium carbonate; (e) solution retarding agents such as paraffin; (f) absorption accelerators such as quaternary ammonium compounds; (g) wetting agents such as cetyl alcohol and glycerol monostearate; (h) absorbents such as kaolin and bentonite clay and (i) lubricants such as talc, calcium stearate, magnesium stearate, solid polyethylene glycols, sodium lauryl sulfate and mixtures thereof.

Other inert diluents can also be present. Diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the core may also include adjuvants such as wetting agents, emulsifying and suspending agents, sweetening, flavoring and perfuming agents.

Suitable non-irritating excipients or carriers such as cocoa butter, polyethylene glycol, or a wax which are solid at one temperature but liquid at another temperature could be used to control the times at which bio-active agent is released as well.

The following example is for purposes of further illustrating the present disclosure. The examples are merely illustrative and are not intended to limit devices made in accordance with the disclosure to the materials, conditions, or process parameters set forth therein.

### Example

### Preparation of Core

Initially, a bio-active agent was adsorbed on inert particles. The bio-active agent was vitamin E, and the particles were CaCO₃. 5 grams of vitamin E was mixed with 5 grams of xylene. 15 grams of CaCO₃ powder was then added, and the three ingredients were mixed together. The particles were dried at ambient temperature for 6 hours, then dried at 50° C. for 4 hours. The result was a free flowing white powder.

5 grams of the powder were mixed with 2 grams of medical grade epoxy (EPO-TEK 301, manufactured by Epo-Tek from USA). The mixture was mixed until a paste was obtained, and molded in a PDMS elastomer mold. The paste was cured for 12 hours at 40° C. and removed from the mold. This formed the core.

### Preparation of Device and Control

The core was then metalized by using electroless plating to apply a copper layer of 5 microns thickness. Two such devices were made.

One device (Device 1) was polished in a corner to remove about 10% of the surface area of the metallic layer.

The second device (Device 2) was not perforated, so the metallic layer was complete.

A third core (Control) was not metallized at all. In other words, this device did not have a metallic layer.

Device 1, Device 2, and Control were placed in paraffin oil for 60 days at 23° C. The content of vitamin E in the paraffin oil was determined by HPLC.

The Control device released 90% of the original vitamin E. Device 2 released less than 1% of the original vitamin E. Device 1 released 20% of the vitamin E.

The results demonstrated the simplicity of loading the bio-active agent into a medical-grade matrix, the good release properties of the core, the good barrier properties of the metallic layer, and the good control of release rate by partially removing the metallic layer.

The present disclosure has been described with reference to exemplary embodiments. Obviously, modifications and alterations will occur to others upon reading and understanding the preceding detailed description. It is intended that the present disclosure be construed as including all such modifications and alterations insofar as they come within the scope of the appended claims or the equivalents thereof.

1. A releasing device for administering a bio-active agent to a living tissue, comprising:
a core comprising a solid organic matrix and at least one bio-active agent contained within the solid organic matrix; and
a metallic layer surrounding the core to form an outer shell;
wherein the core is exposed to its surrounding environment through at least one aperture in the metallic layer of the outer shell thereby, allowing the controlled release of the bio-active agent to the living tissue.

2. The releasing device of claim 1, wherein the metallic layer has a thickness of from about 0.01 micrometers to about 100 micrometers.

3. The releasing device of claim 1, wherein the metallic layer outer surface is a biocompatible metal selected from the group consisting of gold, silver, platinum, stainless steel, and titanium.

4. The releasing device of claim 1, wherein the core has a length of from about 0.1 millimeters to about 50 millimeters.

5. The releasing device of claim 1, wherein the core has a width of from about 10 micrometers to about 50 millimeters.

6. The releasing device of claim 1, wherein the ratio of the aperture area to the total surface area of the device is from 0.01% to 90%.

7. The releasing device of claim 1, further comprising an outer layer, wherein the outer layer encapsulates the metallic layer and comprises an organic binder, a ceramic binder or organo-ceramic binder.

8. The outer layer of claim 8, wherein the outer layer further comprises at least one bio-active compound.

9. A process for producing a releasing device for administering a bio-active agent to a human, animal, or plant patient, comprising:
forming a core having a solid organic matrix containing at least one bio-active agent;
applying a metallic layer to the core to produce an outer shell; and
forming at least one aperture in the metallic layer of the outer shell to allow for the controlled release of the bio-active agent to the patient.

10. The process of claim 9, wherein the core is formed by:
mixing the bio-active agent with a polymer or oligomer or monomer or wax or combination thereof to form a mixture; and
shaping the mixture into a desired shape for the core.

11. The process of claim 10, wherein the polymer or oligomer or monomer or wax or combination thereof is in the form of a melt or a dispersion or an emulsion or a solution.

12. The process of claim 10, wherein the bio-active agent is in the form of a liquid or a solid.

13. The process of claim 10, wherein the bio-active agent is bound to a particle or a polymer prior to mixing to form the mixture.

14. The process of claim 9, wherein the metallic layer is applied by a vapor phase process, an electroless process, or an electroplating process or combinations thereof.

15. The process of claim 9, wherein the at least one aperture is formed by milling, abrading, eroding, etching, or drilling of the metallic layer.

16. The process of claim 9, wherein the at least one aperture is formed by applying an etch resist to the metallic layer, etching at least one aperture into the metallic layer, and removing the etch resist.

17. The process of claim 9, wherein the at least one aperture is formed by applying at least one plating resist to the core, applying a metallic layer to the core, and removing the at least one plating resist to form the at least one aperture in the metallic layer.
